(19) United States
(12) Patent Application Publication
(10) Pub. No.: US 2009/0012506 A1
(43) Pub. Date: Jan. 8, 2009

(54) LASER MASK FOR CREATING A CORNEAL POCKET

(76) Inventor: Vladimir Feingold, Laguna Hills, CA (US)

Correspondence Address: LADAS & PARRY, 5670 WILSHIRE BOULEVARD, SUITE 2100, LOS ANGELES, CA, 90036-5679, US

(21) Appl. No.: 12/281,749

(22) PCT Filed: Mar. 8, 2007
(86) PCT No.: PCT/IB07/04397

§ 371 Date: Sep. 4, 2008

Related U.S. Application Data
(60) Provisional application No. 60/780,365, filed on Mar. 9, 2006.

(51) Int. Cl. A61F 9/01; A61F 9/009

(52) U.S. Cl. 606/5

(57) ABSTRACT

Methods for correcting the vision of a patient by inserting an intracorneal lens into the patient's cornea are provided. The lens is inserted into a corneal pocket that is created by using a mask having an opening with a shape that corresponds to the desired shape of the corneal pocket. A laser ablates tissue within the cornea in an area defined by the shape of the mask since the mask blocks the laser outside the opening. A variety of corneal mask configurations may be used accommodate various corneal lens shapes and sizes.

### CROSS-REFERENCE TO RELATED APPLICATIONS

The present application claims the benefit of U.S. Provisional Patent Application No. 60/780,365, filed on Mar. 9, 2006.

### BACKGROUND OF THE INVENTION

The present invention generally relates to methods of forming a corneal pocket to receive intracorneal refractive lenses and, more particularly to a mask for defining the shape of the corneal pocket receiving the lens.

Intracorneal refractive lenses provide a viable alternative to spectacles and extra-ocular contact lenses for correcting deficiencies in visual acuity and refractive errors. Intracorneal refractive lenses offer a number of advantages for correcting deficiencies in visual acuity. An intracorneal lens may be inserted into an opening in the cornea of an eye having visual abnormalities. Some previous cornea-based techniques have involved the surgical reshaping of inner portions of the cornea to correct visual deficiencies. These include the technique called laser in situ keratomileusis (LASIK). However, such surgical reshaping is not reversible, resulting in some risk of creating permanent visual aberrations for the patient. In contrast, the procedures used with intracorneal lenses are reversible. Also, in typical surgical corneal reshaping surgery an entire flap of the cornea is lifted to permit access for further surgical modification of the cornea. In the surgery used to insert intracorneal lenses, a flap of the cornea is not lifted, but rather a pocket is typically formed in the corneal tissue, which leaves more of the corneal surface intact thereby simplifying healing. Nevertheless, the surgical preparation of such a pocket for an intracorneal lens is difficult to perform accurately.

U.S. Pat. No. 6,599,305 to Feingold discloses a corneal-pocket keratome device to create a corneal pocket and a lens to be inserted and retained in the corneal pocket to effect correction. The corneal-pocket keratome creates a pocket of precise dimensions in the cornea. The corneal-pocket keratome includes a drive unit having cutting head elements which contact the subject eye during corneal pocket formation, and also includes a blade assembly that oscillates laterally while extending forward into the cornea to form the pocket.

Corneal pockets for intracorneal lenses can also be created using electromagnetic radiation, such as a laser. One advantage of laser procedures is that they can ablate tissue within the cornea without the necessity of an incision. Techniques involving laser ablation of intracorneal tissue are taught in U.S. Pat. No. 5,993,438 to Juhasz et al. which discloses an intrastomal photo disruption technique for reshaping the cornea. As disclosed in the Juhasz patent, a pulsed laser beam propagates through corneal tissue and is focused at a point below the surface of the cornea in a layer called the stroma. Laser energy is concentrated in time by ultra short pulse durations and in space by extremely small spot sizes resulting in the creation of very high electric fields that induce a process termed optical breakdown and plasma formation. The ability to reach a subsurface location without necessarily providing a physical pathway allow for the creation of stromal voids or pockets having a desired shape while minimizing the total amount of tissue disrupted.

The application of laser ablation techniques to the creation of corneal pockets is disclosed in U.S. Patent Application Publication No. US 2003/0014042 A1 to Juhasz et al, which discloses techniques for creating a corneal pocket suitable for accommodating a corneal implant using a pulsed laser. Once the corneal pocket is made an entry channel is then created for insertion of the implant.

A number of challenges are presented by the surgical procedures used to create corneal pockets. The creation of a corneal pocket may be a difficult and intricate surgical procedure. Automated processes can be useful in cornea surgery. For example, there are laser surgery systems that can control the depth of the focus of a laser when creating a corneal pocket. However, programmable surgical equipment for controlling the lateral movement to generate the outline of the pocket may not be readily available, or may be prohibitively complicated and expensive for many applications. Some laser surgical systems may be capable of being programmed to control lateral movement, such as those used for LASIK procedures. However, generally such systems are designed for generating corneal flaps and not corneal pockets and may not be easily modified for the creation of a corneal pocket.

As can be seen, there is a need for ways to improve the surgical procedures for creating corneal pockets. There is also a need for improvements in surgical procedures for creating corneal pockets that make the surgery easier, more accurate and faster. There is also a need for a way to assist the surgeon in creating corneal pockets that does not require expensive and complicated programmable equipment. There is also a need for a method for correcting visual abnormalities through surgical implantation of an appropriate corrective lens within the cornea in a precisely predictable and repeatable manner and in such a way that the lens will remain properly positioned and oriented. There is also a need for a method of correcting visual abnormalities which can be reversed and which enables correction of a wide range of visual abnormalities. There is also a need for an efficient method of creating a corneal pocket having different shapes for use with different shaped intracorneal lenses.

### SUMMARY OF THE INVENTION

In one aspect of the present invention, a method for creating an intracorneal cavity comprises the steps of providing a mask having an opening such that the area of the opening corresponds to the shape of a desired intracorneal cavity, the mask being opaque to electromagnetic radiation; positioning the mask in front of a cornea, the cornea having anterior and posterior surfaces; and forming an intracorneal cavity in the cornea by focusing a beam of electromagnetic radiation to a predefined depth inside the cornea between the anterior and posterior surfaces such that the electromagnetic radiation ablates corneal tissue at the predefined depth, and moving the beam of electromagnetic radiation within the area of the mask opening to create an intracorneal cavity having substantially the same shape as the area of the opening.

In another aspect of the present invention, a method for correcting the vision of a patient comprises the steps of providing an intracorneal lens; providing a mask having an opening defining the outline of a corneal pocket, the mask being opaque to electromagnetic radiation; positioning the mask in front of the cornea of the patient, the cornea having anterior and posterior surfaces; forming a corneal pocket in the cornea by focusing a beam of electromagnetic radiation to a predefined depth inside the cornea between the anterior and posterior surfaces, the electromagnetic radiation ablating corneal tissue at the predefined depth; moving the beam of electromagnetic radiation across the area of the opening, wherein the mask prevents the ablation of tissue outside of the outline of the corneal pocket; forming an entry channel in the cornea through the anterior surface of the cornea to the predefined depth in communication with the pocket; and inserting the intracorneal lens into the corneal pocket.

In a further aspect of the invention, there is provided a mask for creating a corneal pocket comprising a member having an opening, the member being opaque to some wavelengths of electromagnetic radiation; and the opening defining the outline of a corneal pocket.

### BRIEF DESCRIPTION OF THE DRAWINGS

FIG. 1 is a simplified schematic diagram of an apparatus for performing laser surgery in accordance with an embodiment of the invention;

FIG. 2 is a sectional view of a corneal pocket according to an embodiment of the present invention;

FIG. 3 is a sectional view of the anterior portion of an eye having an implant disposed within the cornea of the eye, according to an embodiment of the invention;

FIG. 4 schematically represents a series of steps involved in a method for inserting an lens in the cornea of a patient, according to another embodiment of the invention;

FIGS. 5A-5M depict top views of exemplary configurations of a mask for creating corneal pockets according to various embodiments of the present invention; and

FIGS. 6A-6M depict top views of exemplary configurations of corneal pockets created using the masks shown in FIGS. 5A-5M.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention relates to methods for correction of a visual deficiency of a patient. The present invention also relates to methods for using a mask to create an intracorneal pocket for receiving an intracorneal lens in a patient's eye. The present invention still further relates to a variety of mask configurations to create a variety of corneal pocket configurations.

In contrast to the prior art, some embodiments of the present invention use masks to insure that intracorneal pockets created by a laser conform to a desired shape. This enables the surgeon to accurately and quickly control the shape of the pocket without requiring costly and complicated programmable equipment. Also, in some embodiments of the present invention, a variety of different corneal pocket shapes may be created to further enhance the ease of insertion of a intracorneal lens of different shapes and sizes, and to maximize the surface of the cornea which is left intact, thereby facilitating healing. Other surgical procedures such as the creation of arcuate cuts on the cornea may also be made using the masks in accordance with some embodiments of the invention.

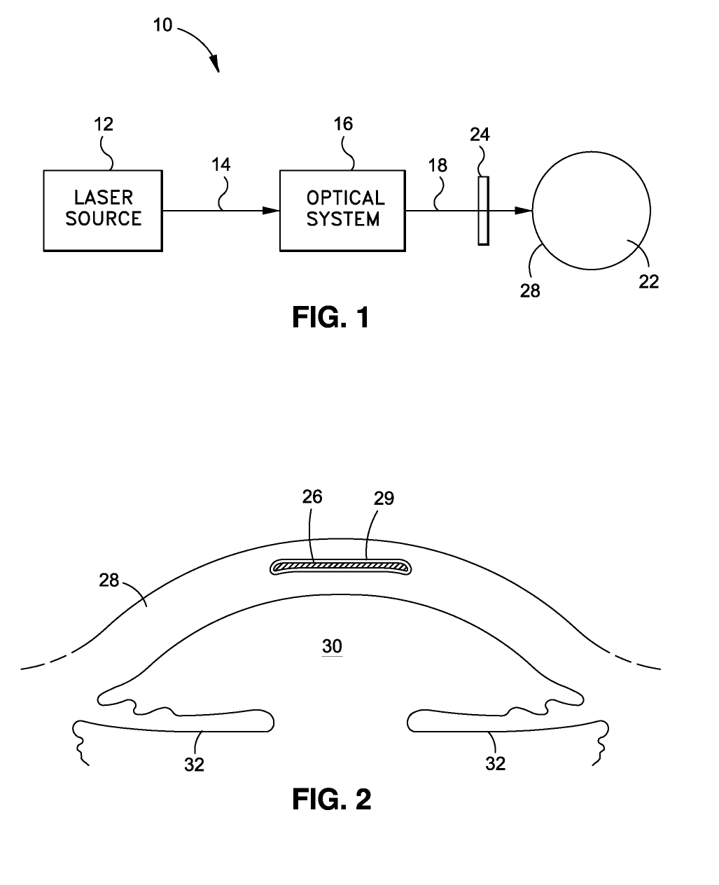

FIG. 1 shows a laser surgery apparatus 10 for laser surgery to create an intracorneal pocket in accordance with an embodiment of the invention. The laser surgery apparatus 10 may include a laser source 12 which preferably may be capable of generating and controlling a source beam 14 having a continuous train of laser pulses of substantially constant pulse duration and pulse energy. In one embodiment of the laser surgery apparatus 10, a source beam 14 may have a pulse duration of between approximately 500 picoseconds and 10 femptoseconds, a wavelength longer than 800 nanometers and pulse energy of approximately 6 μJ.

The laser surgery apparatus 10 further includes an optical system 16 for forming a shaped laser beam 18 and directing the shaped laser beam 18 toward and into the cornea 28 of an eye 22. Also shown in FIG. 1 is a mask 24 which is used to control the area of the cornea that is exposed to the laser beam 18 as described in more detail below.

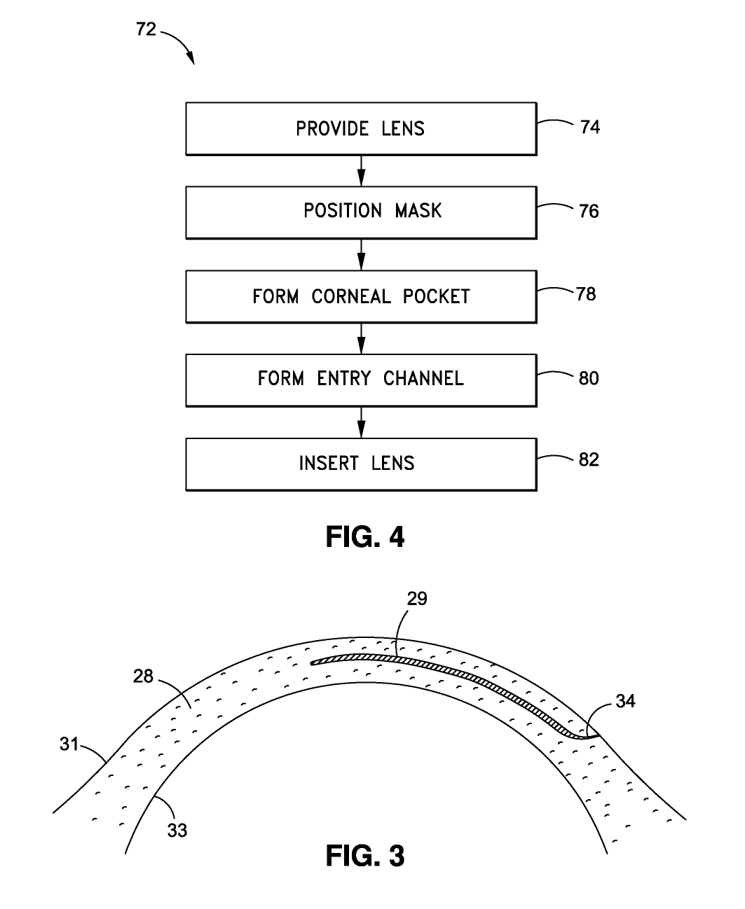

FIG. 2 is a sectional view of the anterior portion of the eye 22 having an intracorneal lens 26 disposed therein, according to an embodiment of the invention. In the embodiment of the invention shown in FIG. 2, intracorneal lens 26 may be disposed within a cornea 28 of the eye 22, which may partially enclose the anterior chamber 30 of the eye 22. Also shown in FIG. 2 is an iris 32. In accordance with an embodiment of the invention, lens 26 may be inserted within cornea 28 following formation of a corneal pocket 29, which may be formed using a laser surgery apparatus 10 as shown in FIG. 1.

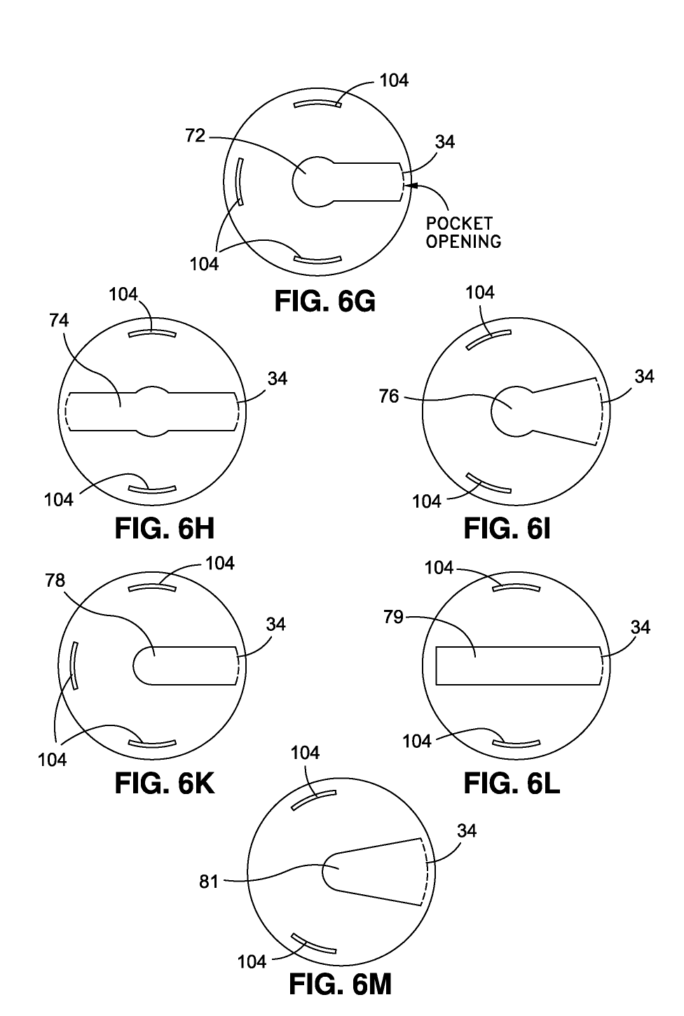

Intracorneal lens 26 is not restricted to the configuration shown in the drawings, but may have various shapes, such as circular or oval. In some embodiments, intracorneal lens 26 may have a doughnut-like configuration. The size and shape of intracorneal lens 26 may, in some cases, determine the size and shape of the corneal pocket. Various embodiment of such cornea pockets are described below and shown in FIGS. 6A-6F.

The intracorneal lens 26 preferably may be formed of a biocompatible material that permits sufficient gas diffusion to allow adequate oxygenation of internal eye tissues. Such materials may include silicone, hydrogels, urethanes or acrylics. It also may be desirable that the lens be made of a hydrophilic material which swells somewhat when hydrated. Such materials, for example, hydrogels, are well known and are used in some present contact lenses.

The optical characteristics of intracorneal lens 26 may be selected for correcting various visual deficiencies, including without limitation: myopia (short sightedness), hypermetropia (long sightedness), presbyopia and astigmatis. As an example, intracorneal lens 26 may have a diopter power or value in the range of from +15 to −30. Intracorneal lens 26 may be customized for a particular patient to provide optical characteristics to correct a specific visual defect of a patient. Intracorneal lens 26 may be multi-focal, may be provided as an off-the-shelf unit with pre-determined optical characteristics and may have zones with optical power and zones without optical power. It is to be understood that the present invention is not limited to treatment of the aforementioned visual defects, and that treatment of other eye conditions is also within the scope of the invention.

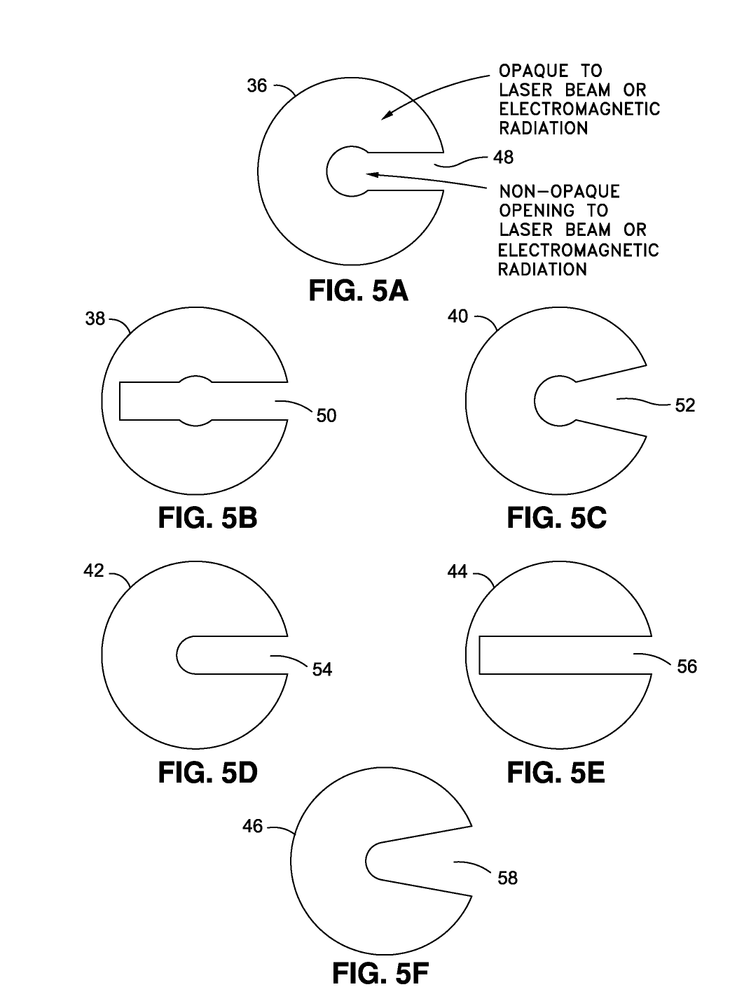

FIG. 3 shows a cross section of a cornea 28 having a corneal pocket 29 formed by a laser surgery apparatus 10 in accordance with one embodiment of the invention. Cornea 28 has an anterior surface 31 and a posterior surface 33. Corneal pocket 29 may be formed by photo ablation using laser beam 18 from a laser source 12. The techniques for creating a corneal pocket are well known and are described in U.S. Pat. No. 7,101,364 to Bille and U.S. Patent Application Publication No. US 2003/0014042 A1 to Juhasz et al. The contents of both of these documents are incorporated by reference in their entirety.

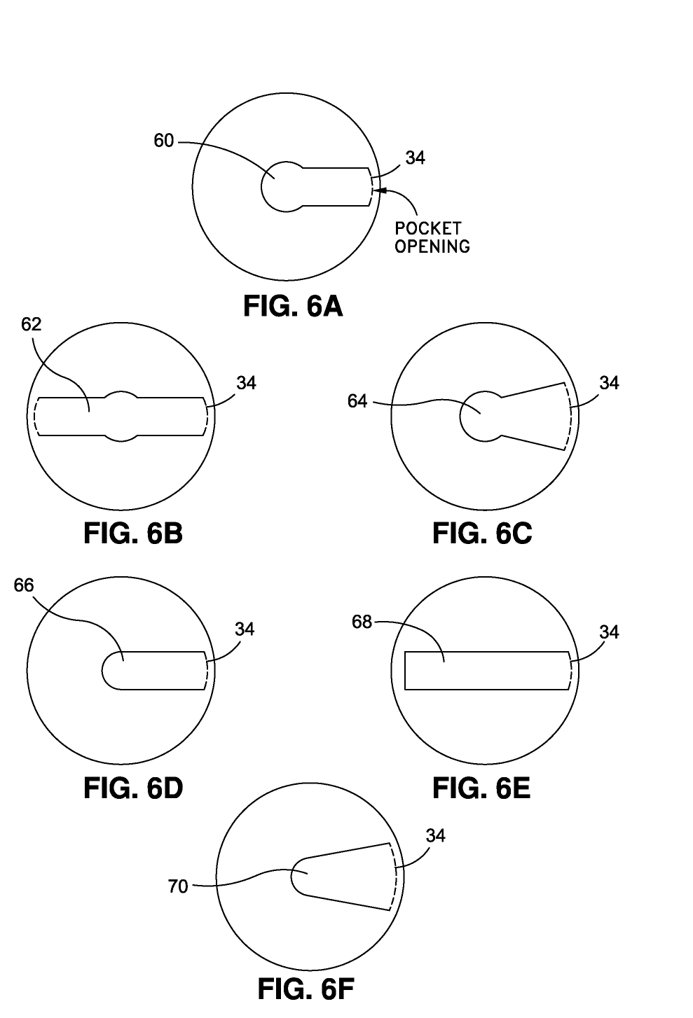

The corneal pocket 29 may be formed with a thickness and shape that conforms to the surfaces of the intracorneal lens 26. For example, the interior surfaces of the corneal pocket 29 may be convex, concave, planar or irregular. The edges of the corneal pocket 29 may form an outline having various shapes depending on the desired outcome and the shape of the intracorneal lens 26. The mask 24 shown in FIG. 1 may prevent the laser beam 18 from ablating tissue beyond the boundaries of the desired shape. FIGS. 5A-5M show exemplary masks which may be used for this purpose. In particular, masks 36, 38, 40, 42, 44, 46, 80, 82, 84, 86, 88 and 90 may be made of a material that is opaque to the laser beam 18 from laser source 12. Each mask may have an open region 48, 50, 52, 54, 56, 58, 92, 94, 96, 98, 100 and 102 which may be transparent to the laser beam 18 from laser source 12. As shown in FIGS. 5G-5M are arcuate openings 103. Open regions 48, 50, 52, 54, 58, 92, 96, 98, 100 and 102 may include both straight portions and arced portions. Open regions 56 and 94 may include straight portions extending across most of the surface of the masks 44 and 82 respectively.

FIGS. 6A-6M show the resulting corneal pockets 60, 62, 64, 66, 68, 70, 72, 74, 76, 78, 79 and 81 that may be created by the laser beam 18 passing through open regions of the corresponding masks 36, 38, 40, 42, 44, 46, 80, 82, 84, 86, 88 and 90. Also shown in FIGS. 6G-6M are the arcuate cuts 104 that may be created as a result of the laser beam 18 passing through arcuate openings 103. The various configurations of corneal pockets shown in FIGS. 6A-6M may be adapted to be used with lenses of various shapes and sizes. The corneal pockets 60-81 as configured may also facilitate the insertion of the lens and minimize the size of the incision for improved post-surgical healing of the cornea.

All of the corneal pockets shown in FIGS. 6A-6M may include an entry channel 34 that may be cut into the cornea 28 after the corneal pocket 29 is formed. Entry channel 34 may permit the insertion of the intracorneal lens 26 into the corneal pocket 29.

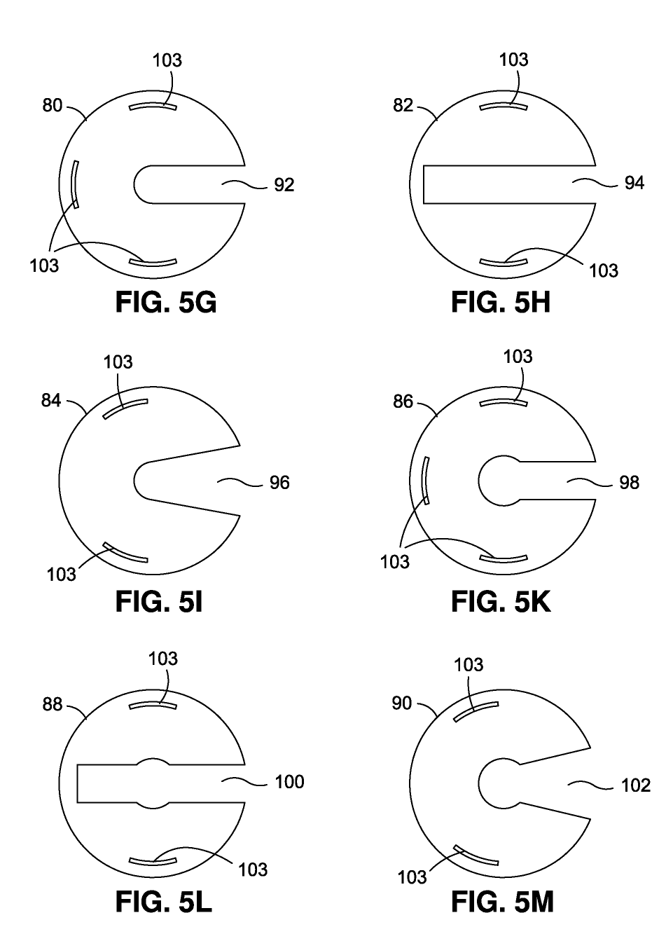

FIG. 4 schematically represents a series of steps involved in a process 72 for creating a corneal pocket and inserting a lens in the cornea of a patient, according to one embodiment of the invention. The process 72 may begin with the step 74 of providing an intracorneal lens 26. The intracorneal lens 26 may or may not have optical power depending on the purpose of the intracorneal lens 26. In step 76 a mask 24 may be positioned in front of the cornea 28. Mask 24 may have one of the configurations shown in FIG. 5A-F, or may have a different configuration. In step 78 a corneal pocket 29 may be formed. This may be done using the laser surgery apparatus 10 shown in FIG. 1. In particular, a laser source 12 being controlled by an optical system 16 may be used to focus a laser beam 18 within the corneal tissue. The laser beam 18 will ablate a region of the cornea tissue in the area of the focus of the laser beam 18. The focus of the laser beam 18 may then be moved laterally by hand to cut a layer of corneal tissue. While the focus of the laser beam 18 is being moved laterally, it may be maintained a fixed depth within the cornea using known laser surgical techniques. The focus of the laser beam 18 may be easily, quickly and accurately moved laterally by hand within the confines of the mask open region without the risk of cutting outside the desired area defined by the mask. In this way, the masks shown in FIGS. 5A-5M may be used to create the corneal pockets shown in FIGS. 6A-6M.

The thickness of the corneal pocket created using the above techniques will be about the size of the diameter of the laser beam 18 focal point. In some cases, depending on the thickness and shape of the intracorneal lens 26, additional tissue may be ablated at different depths within the cornea 28.

In step 80 an entry channel 34 may be formed. This may be accomplished using the laser source 12 or may be formed using a conventional scalpel. Entry channel 34 may provide a means for insertion of the intracorneal lens 26 and also will allow the release of gasses created by laser ablation when the intracorneal pocket 29 is formed.

The intracorneal lens 26 may then be inserted into the intracorneal pocket 29 in step 82. Step 82 may further involve temporarily deforming the intracorneal lens 26 before it is introduced into the cornea 28. The intracorneal lens 26 may be deformed by rolling, folding, and the like. The intracorneal lens 26 may have prescribed memory characteristics that allow it to return to its original size and configuration after insertion into the cornea 28, while retaining its desired optical characteristics. The intracorneal lens 29 may be made of a hydrophilic material which swells when hydrated. The lens may be inserted fully hydrated to elastically fit into a corneal pocket, or while at least partly dehydrated such that subsequent hydration helps secure the fit in the pocket.

As can be appreciated by those skilled in the art, the present invention may provide a method for correcting the vision of a patient with an intracorneal lens 26 that may be easily inserted into a corneal pocket 29. The corneal pocket 29 may be created using a laser source 12 or may be created using other forms of electromagnetic radiation. The creation of the corneal pocket 29 is facilitated by the use of a mask 24 that prevents the laser beam 18 from ablating tissue outside the boundary of a desired shape. The mask 24 may also be used in other kinds of ophthalmic surgery requiring partial lamellar resection, including LASIK surgery. A variety of corneal pocket configurations may be used to accommodate various corneal lens shapes and sizes. Other surgical procedures, such as arcuate cuts, may also be made using the techniques of the invention.

It should be understood, of course, that the foregoing relates to exemplary embodiments of the invention and that modifications may be made without departing from the spirit and scope of the invention as set forth in the following claims.

We claim:

1. A method for creating an intracorneal cavity comprising:
providing a mask having an opening such that the area of said opening corresponds to the shape of a desired intracorneal cavity, said mask being opaque to electromagnetic radiation;
positioning said mask in front of a cornea, said cornea having anterior and posterior surfaces; and
forming an intracorneal cavity said cornea by focusing a beam of electromagnetic radiation to a predefined depth inside said cornea between said anterior and posterior surfaces such that said electromagnetic radiation ablates corneal tissue at said predefined depth, and moving said beam of electromagnetic radiation within said area of said mask opening to create an intracorneal cavity having substantially the same shape as said area of said opening.

2. The method of claim 1 further comprising forming an entry channel in said cornea through the anterior surface of said cornea to said predefined depth.

3. The method of claim 1 wherein said intracorneal cavity is a corneal pocket.

4. The method of claim 3 wherein said intracorneal cavity is covered by a corneal flap.

5. The method of claim 1 wherein said shape of a desired intracorneal cavity includes at least one straight portion.

6. The method of claim 1 wherein said shape of a desired intracorneal cavity includes at least one curved portion.

7. The method of claim 1 wherein said shape of a desired intracorneal cavity includes at least one straight portion and at least one curved portion.

8. The method of claim 1 wherein said shape of a desired intracorneal cavity includes a pair of straight portions.

9. The method of claim 8 wherein said pair of straight portions are parallel to each other.

10. The method of claim 1 wherein said electromagnetic radiation is emitted from a laser.

11. The method of claim 1 wherein said focal point has a known diameter and the thickness of said corneal pocket is substantially the same as said focal point diameter.

12. The method of claim 11 further comprising repeating the step of moving said beam of electromagnetic radiation within the area of said opening at a second predetermined depth such that the thickness of said corneal pocket is greater than said focal point diameter.

13. A method for correcting vision of a patient, comprising:
providing an intracorneal lens;
providing a mask having an opening defining the outline of a corneal pocket, said mask being opaque to electromagnetic radiation;
positioning said mask in front of the cornea of said patient, said cornea having anterior and posterior surfaces;
forming a corneal pocket in said cornea by focusing a beam of electromagnetic radiation to a predefined depth inside said cornea between said anterior and posterior surfaces, said electromagnetic radiation ablating corneal tissue at said predefined depth;
moving said beam of electromagnetic radiation across the area of said opening, wherein said mask prevents the ablation of tissue outside of said outline of said corneal pocket;
forming an entry channel in said cornea through the anterior surface of said cornea to said predefined depth in communication with said pocket; and
inserting said intracorneal lens into said corneal pocket.

14. The method of claim 13 wherein said lens is a refractive lens.

15. The method of claim 13 further comprising repeating the step of moving said beam of electromagnetic radiation within the area of said opening at a second predetermined depth such that the thickness of said corneal pocket is greater than said focal point diameter.

16. The method of claim 15 further comprising repeating the step of moving said beam of electromagnetic radiation within portions of the area of said opening at additional predetermined depths such that the thickness of said corneal pocket varies and is substantially the same as thickness of the lens over corresponding areas of the lens.

17. A mask for creating a corneal pocket comprising;
a member having an opening, said member being opaque to some wavelengths of electromagnetic radiation; and
said opening defining the outline of a corneal pocket.

18. The mask of claim 17 wherein said opening in said mask includes an arced portion and a straight portion adjacent the arced portion.

19. The mask of claim 18 wherein said straight portion includes a straight portion extending radially away from said arced portion.

20. The mask of claim 19 wherein said straight portion further comprises first and second straight portions extending radially from said arced portion and being disposed on opposite sides of said arced portion.

21. The mask of claim 18 wherein said straight portion comprises parallel edges extending radially away from said arced portion.

22. The mask of claim 18 wherein said straight portion comprises nonparallel edges extending away from said arced portion.

23. The mask of claim 17 wherein said opening includes a straight portion extending across the cornea.

24. The mask of claim 17 further comprising at least one arcuate opening.
